(19) United States
(12) Patent Application Publication
(10) Pub. No.: US 2013/0066213 A1
(43) Pub. Date: Mar. 14, 2013

(54) EYE MONITOR

(71) Applicant: Iain Tristan Wellington, Rochester (GB)

(72) Inventor: Iain Tristan Wellington, Rochester (GB)

(21) Appl. No.: 13/698,855

(22) PCT Filed: May 17, 2011
(86) PCT No.: PCT/GB11/50941

§ 371 Date: Nov. 19, 2012

(51) Int. Cl. A61B 3/11

(52) U.S. Cl. CPC A61B 3/11
USPC 600/475

(57) ABSTRACT

An eye monitor is disclosed for monitoring the size of a pupil of a monitored subject. The eye monitor includes a tracking device for tracking movement of the user's pupil and a monitoring device for monitoring the size of the user's pupil. The eye monitor provides for a continuous and real time analysis of the subject's eye and in particular the pupil of the eye, to determine whether the subject has been exposed to harmful chemicals and without requiring the subject to actively participate in the monitoring.

The present invention relates to an eye monitor or method of monitoring pupillary response, in particularly, but not exclusively, to an eye monitor for monitoring the size of a pupil of an eye of a monitored subject.

U.S. Pat. No. 6,637,885 discloses a method for the self-detection of exposure to chemicals such as organophosphates and harmful gas, by monitoring pupillary response. The method disclosed requires a user to place an eyeglass cup of a monitoring device over the eye to be tested, blocking the light from entering the other eye and to subsequently observe whether the pupil of the eye under test dilates upon switching the device on.

The device operates on the basis that the pupil of the eye being blocked will tend to dilate, due to reduced levels of light entering the blocked eye. The pupil of the eye under test should respond consensually, i.e. the pupil of the eye under test should also dilate. If the pupil of the eye under test does not dilate then it is possible that miosis (pupil contraction) has occurred, which is indicative of exposure to chemicals.

However, a problem with the device disclosed in U.S. Pat. No. 6,637,885 is that a user must perform frequent checks to determine whether they have been exposed to dangerous gases, and as such, the user must stop performing their current task to carry out the check. This can be a particular problem, particularly for aircraft pilots. In addition, since a pilot may travel large distances in a relatively short time, then it would be necessary to perform the checks more frequently to ascertain whether they have been exposed to dangerous chemicals.

In addition, the device of U.S. Pat. No. 6,637,885 requires a user to cover both eyes for a period of time. This is undesirable when flying an aircraft or during military manoeuvres, when it is necessary to remain alert and vigilant, for example.

In accordance with a first aspect of the present invention, there is provided eye monitor for monitoring the size of a pupil of an eye of a monitored subject, the monitor comprising: an infra-red transmitter for transmitting an infrared radiation probe signal substantially upon the eye of the subject; an infrared sensor for sensing variations in intensity of the infrared radiation reflected from the eye of the subject and outputting a sensor signal in accordance with the sensed variations; a processor arranged to receive said sensor signal and determine a location of a pupil of a subject dependent on the sensed variations; and signal steering means arranged to scan the probe signal across the eye of the subject responsive to said processor to direct the probe signal at the pupil of the subject, wherein the processor is arranged to determine the size of the pupil dependent on the sensed variations.

In this manner, the detection of the exposure of a subject to harmful substances or chemicals can be undertaken without active input from the subject. The ability of the eye monitor to track the pupil of an eye of a subject provides for a continuous, real time analysis of the pupil to determine whether the subject has been exposed to harmful toxins, for example, and thus can enable a warning to be issued to the subject to take the appropriate action when exposure has occurred.

The eye monitor may comprise means for locating the transmitter and sensor relative to a monitored eye for monitoring the size of a pupil. In this way, the user is not required to position the monitor in place relative to a pupil for testing.

The processor may be configured to determine the size of a pupil at intervals over a monitoring period. In one arrangement, a frequency of the intervals may be dependent on a velocity of the subject.

The processor may be configured to determine the size of a pupil generally continuously over a monitoring period.

The monitoring period may be selected for monitoring a subject over a risk period during which the subject is potentially exposed to harmful ambient agents.

A memory may be provided for storing calibrated pupil size. In this case, the processor is arranged to compare said determined pupil size with said calibrated size and determines exposure to harmful ambient agents when the determined size is inconsistent with the calibrated pupil size.

In use of one arrangement, the intensity of the infrared radiation reflected from the pupil of the subject is relatively low and the intensity of the infrared radiation reflected from a region of the eye around the pupil of the subject is relatively high, and the processor is configured to determine a location of the pupil by determination of a location of said relatively low reflected variation and to determine the size of the pupil by determination of the distance in at least one dimension across the eye of said relatively low reflected radiation.

The eye monitor may further include control means for controlling the operation of the transmitter and the receiver. The control means and/or the processor may be communicatively coupled with the transmitter and the receiver. The control means and the processor may be disposed upon the eye monitor or upon a pilot's helmet, for example.

According to another aspect of the invention, a head or helmet mountable eye monitor includes an eye monitor mounted upon a frame, for example, a frame for a pair of spectacles. The frame may be arranged to support an optical element in front of one or both of the eyes of the subject. The optical element may include a lens, visor or optical combiner.

According to another aspect of the invention, a vehicle mountable eye monitor includes an eye monitor mounted to the structure of a vehicle. For example, the eye monitor may be mounted within an aircraft cockpit, a land vehicle such as a tank, armoured vehicle or other transport vehicle such as a passenger bus, train or aircraft.

In accordance with another aspect of the present invention, there is provided a method of monitoring the size of a pupil of an eye of a monitored subject, the method comprising: transmitting an infrared radiation probe signal substantially upon the eye of the subject; sensing variations in intensity of the infrared radiation reflected from the eye of the subject and outputting a sensor signal in accordance with the sensed variations; determining a location of a pupil of a subject dependent on the sensed variations; scanning the probe signal across the eye of the subject responsive to the determined location of the pupil of the subject; and determining the size of the pupil dependent on the sensed variations.

The intensity of the infrared radiation reflected from the pupil of the subject may be relatively low and the intensity of the infrared radiation reflected from a region of the eye around the pupil of the subject may be relatively high, and the method may comprise determining a location of the pupil by determining a location of said relatively low reflected variation and determining the size of the pupil by determining the distance in at least one dimension across the eye of said relatively low reflected radiation.

The invention also provides such a method for detecting exposure of a subject to harmful ambient agents.

The method may include comparing the measured pupil size with a calibrated acceptable pupil range.

An embodiment of the present invention will now be described by way of example only and with reference to the accompanying drawings, in which:

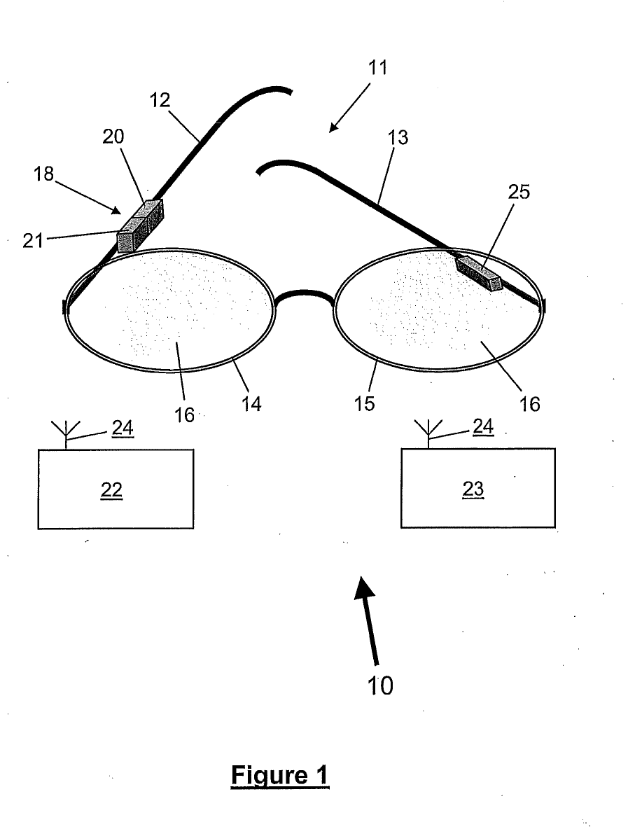

FIG. 1 is a schematic representation of the head mountable eye monitor according to an embodiment of the present invention;

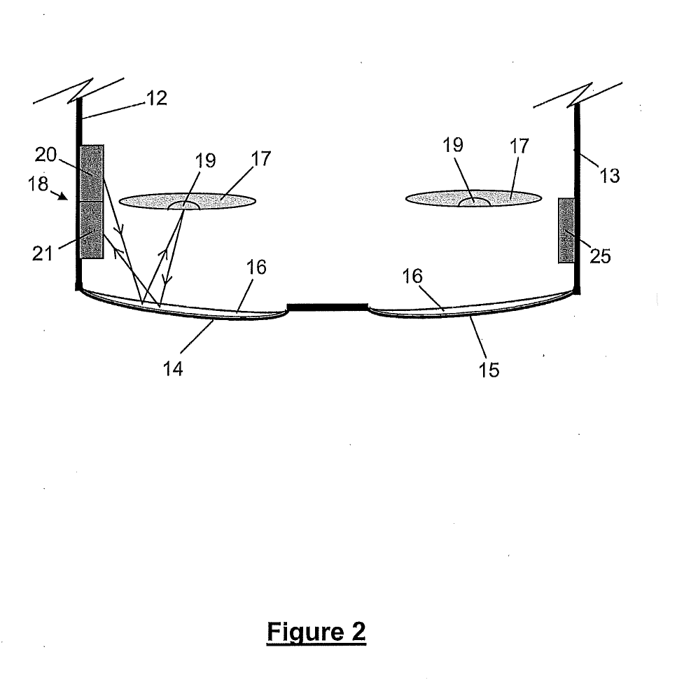

FIG. 2 is a plan view of the head mountable eye monitor illustrated in FIG. 1, showing the path of the transmitted and reflected probe signal;

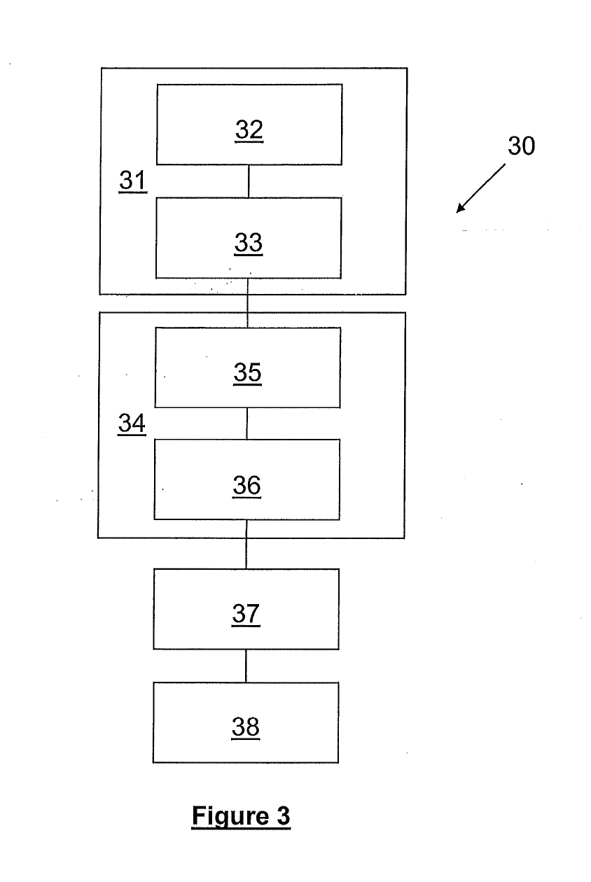

FIG. 3 is a flowchart illustrating the steps associated with a method of monitoring pupillary response according to an embodiment of the present invention;

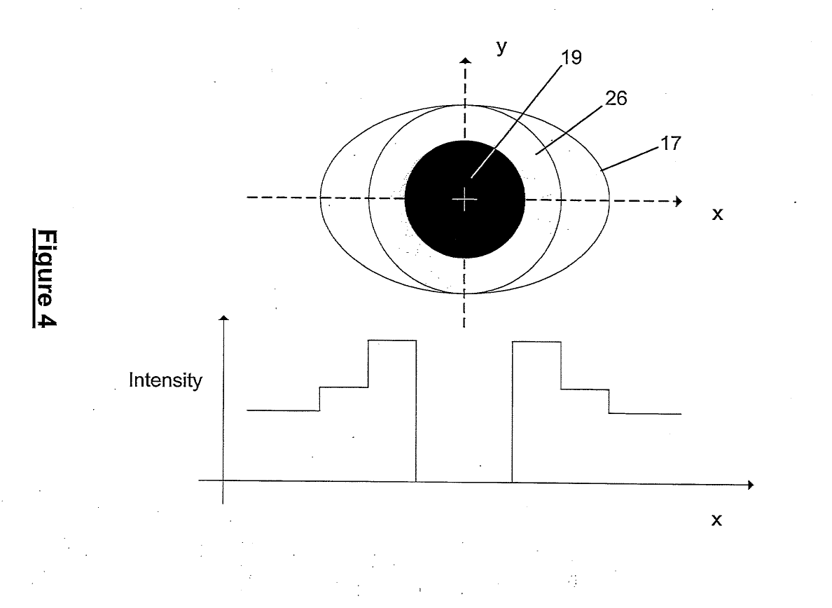

FIG. 4 is a graphical representation of the variation of the signal intensity reflected from positions across a user's eye;

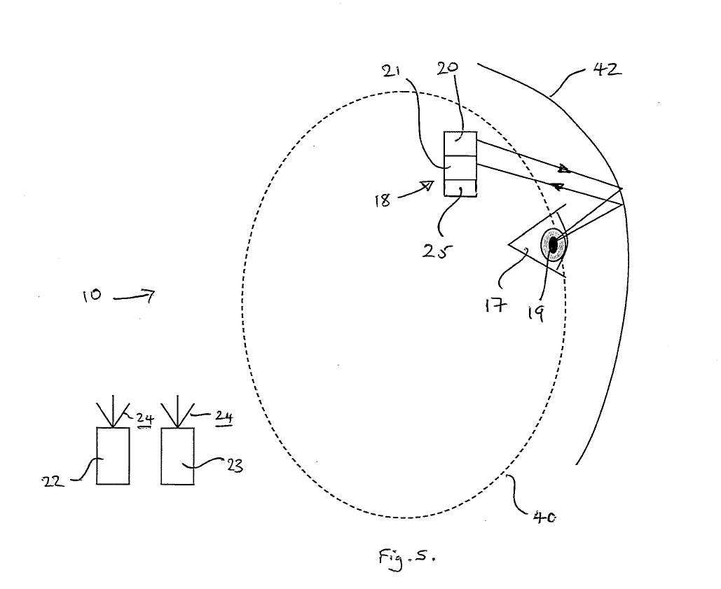

FIG. 5 is a schematic representation of a helmet mountable eye monitor according to an embodiment of the present invention; and

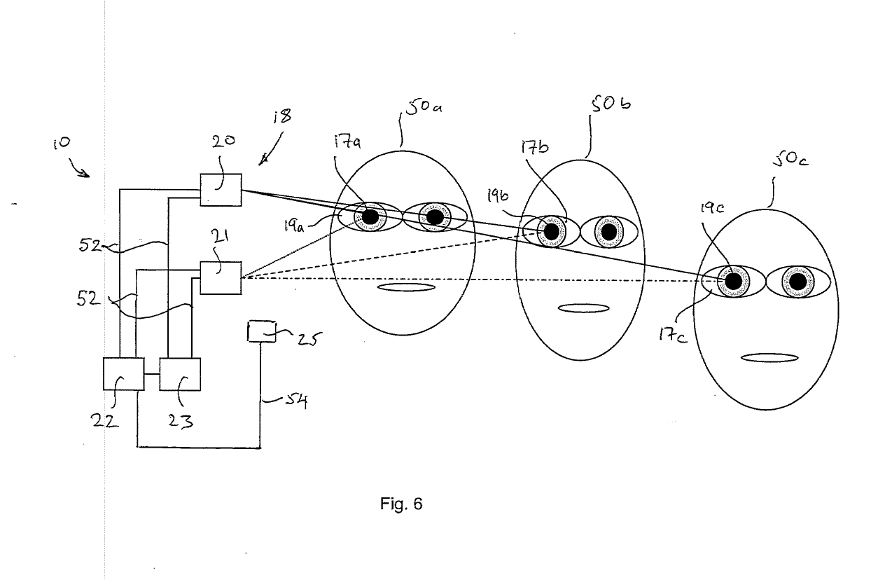

FIG. 6 is a schematic representation of a vehicle mountable eye monitor according to an embodiment of the present invention.

Referring to FIGS. 1 to 4, wherein like references have been used to indicate similar integers, there is illustrated a head mountable eye monitor 10 according to an embodiment of the present invention, which obviates the requirement for a subject being monitored, in this case a user (not shown), to cover both eyes in determining whether the user (not shown) has been exposed to harmful chemicals. The eye monitor 10 comprises a frame 11, such as a frame for a pair of spectacles, having a first and second arm 12, 13 which extend from a first and second support 14, 15 for an associated optical element 16, such as a lens. The arms 12, 13 are separately hingedly coupled at a proximal end thereof, to the respective optical element support 14, 15, which are separately arranged to support the associated optical element 16 which is arranged to be substantially in front of each of the user's eyes 17 when in use. It is envisaged that the optical elements 16 may be coated with a reflection coating (not shown) to prevent pilots (not shown) for example, from becoming blinded by laser pointers (not shown), for example.

The frame 11 is further arranged to support a tracking arrangement 18, which is arranged initially to locate a centre position of a pupil 19 of one or both of the user's eyes 17 and subsequently to autonomously track the movement of the user's eyes 17, and in particular movement of the pupils 19 of the user's eyes 17. The following description will refer to the tracking and monitoring the pupil 19 of one of the user's eyes 17, however, the skilled reader will recognise that the monitor of the present invention could also be used to track and monitor the pupil 19 of each of the user's eyes 17.

The tracking arrangement 18 comprises a transmitter 20, which is arranged to transmit an infrared probe signal substantially upon a user's eye 17 and a receiver 21, such as an infrared camera or sensor, which is arranged to receive an infrared signal which becomes reflected off the user's eye 17. The tracking arrangement 18 further comprises signal steering means (not shown) which is arranged to suitably direct the signal upon the user's eye 17 and to scan the probe signal across the user's eye 17.

The eye monitor 10 further comprises a processor 22 for processing signals received from the receiver 21 and a control unit 23 for controlling the operation of the transmitter 20 and receiver 21. The processor 22 and control unit 23 are communicatively coupled with the tracking arrangement 18 and may be supported upon the frame 11 or otherwise disposed within a cockpit (not shown) or upon a pilots helmet (not shown), for example. In the embodiment illustrated in FIG. 1, the processor 22 and control unit 23 are arranged in communication with the tracking arrangement 18 via a wireless communication link 24. The eye monitor 10 may further comprise a light sensor 25 mounted upon the frame 11 for example for monitoring the ambient light conditions, and is communicatively coupled with the processor 22 via the wireless communications link 24, for example.

When a person is exposed to certain harmful ambient agents, including chemical, biological or nuclear agents, pupillary response is typically abnormal. The present eye monitor is configured to detect such abnormal response. The abnormal response may be pupillary dilation or constriction, to an extent in either case which is over and above expected dilation or constriction. Therefore, the eye monitor may be configured to detect pupillary dilation or constriction over a predetermined amount above which exposure to harmful agents is deemed to have taken place. The predetermined amount may be determined by calibration of an individual pilot or other person, or may be estimated.

Under normal conditions, a pupil of a person will almost continually dilate and constrict in response to for example small changes in ambient light or during focusing. When exposed to harmful agents, a pupil may become fixed in dilation or constriction without normal fluctuations in pupil size. The present eye monitor may be configured to determine when the size of a pupil remains fixed over a selected period and therefore that exposure to harmful agents has occurred. Since a pupil will remain of constant size for relatively short times during normal behaviour, the period selected should be sufficient long to avoid an incorrect determination. The eye monitor may be configured to determine pupil size at intervals of the selected period.

In an aircraft, a pilot will typically alternate between viewing the outside world scene through a canopy or windshield and viewing the inside of a cockpit. Given that during daylight, the outside world scene is relatively bright and the cockpit is relatively dark, or at night, the outside world scene may be relatively dark whilst the cockpit is relatively light, a change in pupil size occurs when a pilot changes view between outside and inside. In for example military aircraft, the orientation of a pilots head is typically monitored so that a head up or head mounted display can display to the pilot information relating to the pilots line of sight. Accordingly, the head monitoring equipment may supply to the processor of the eye monitor head movement information indicative of a change in the pilot's line of sight between outside and inside. The eye monitor may be arranged to take pupillary readings shortly before and shortly after head movement, and determine if pupillary response is abnormal given the change of head orientation.

In another method, the eye monitor may sense ambient light and to determine if expected pupillary response occurs in response to changes in ambient light. For example, the eye monitor may be configured to determine pupil size sufficiently regularly so that a pupil size shortly after a change in ambient light can be compared with the pupil size shortly prior to the change.

It should be noted that in many known methods, an eye is stimulated by a light source and the pupillary reaction is measured. In some known methods, one or both eyes must be covered for testing meaning that at least for testing a person cannot use their eyes for seeing. Clearly this is disadvantageous for a pilot. These known methods measure pupil size, but do not monitor pupil size over a prolonged period whilst a person is engaged in other visual activity. The present eye monitor allows a pupil to be monitored continuously or at regular intervals without discontinuing their activity over a period during which a person is considered to be at risk. Monitoring may be initiated by a person wearing the eye monitor or may be initiated remotely, particularly if a plurality of people are wearing eye monitors and are subjected to risk generally simultaneously.

Referring to FIG. 3 of the drawings, there is illustrated a flowchart of the steps associated with a method 30 of monitoring pupillary response according to an embodiment of the present invention. In order to track and monitor the pupil of a user's eye 17, the tracking arrangement 18 is arranged to first locate the position of the centre of the user's pupil. This is performed during a pupil location step 31 in which the probe signal is scanned across the user's eye 17 at step 32 and the variation in reflected signal intensity in the x-direction and y-direction is monitored at step 33. The location of the centre position of the pupil 19 of the user's eye 17 is achieved by directing the infrared probe signal upon the optical element 16 disposed in front of the respective eye 17, such that the probe signal becomes reflected off the optical element 16 onto the respective pupil 19. The probe signal, which subsequently becomes reflected off the pupil 19 is further arranged to reflect off the optical element 16 onto the receiver 21. The receiver 21 subsequently communicates the received signal data via the wireless communications link 24 to the processor 22, which is arranged to determine the size of the pupil 19. The processor 22 further receives data from the light sensor 25 relating to the ambient lighting levels. The centre-point of the pupil position is determined by identifying the intersection of the x-axis and y-axis. A typical graphical representation of the variation in reflected light intensity is illustrated in FIG. 4 of the drawings.

Once the location of the centre position of the pupil 19 has been determined, the tracking arrangement 18 is subsequently arranged to track the movement of the user's pupil 19 at step 34. This is achieved by monitoring for changes in the intensity of the reflected signal, as the user moves their eye 17. As the tracking arrangement 18, tracks the movement of the user's eye 17, the probe signal is directed by the signal steering means (not shown) upon the user's eye 17 at step 35, and the processor 22 is arranged to process the signal reflected from the user's eye 17 to determine the size of the user's pupil 19 at step 36. This is performed by determining the range either side of the centre-point, which provides substantially the same reflected signal intensity, and may take place at a rate of 10-100 times per second. The signal reflected off the user's eye 17 is dependent upon the position on the eye 17 from which the signal becomes reflected from. The pupil 19 and iris 26 which surrounds the pupil, reflect the signal by different amounts. Accordingly, once the centre-point of the pupil has been identified, the pupil diameter can be measured by determining the range either side of the centre-point which provides substantially the same reflected signal intensity. The algorithm used by the processor 22 is arranged to provide a time average of the determined pupil size so that a reduction in error or false measurements can be achieved. Accordingly, by constantly monitoring the size of the user's pupil 19, a comprehensive record of the range of pupil sizes can be determined, which therefore allows for a rapid identification of unusual pupil activity.

Alternatively, the determined pupil size or time average pupil size may be compared with a calibrated range of acceptable pupil sizes. The calibrated range may be obtained by monitoring the range of pupil sizes for the user (not shown) under various controlled lighting conditions. As a further alternative, the processor 22 may be used to compare the determined pupil size with an average pupil diameter for a population group to which the user (not shown) belongs and for that particular lighting level.

If the measured pupil size is found to reside outside of an acceptable range at step 37, the head mountable eye monitor 10 is arranged to warn the user, such as the pilot (not shown), via an audible and/or visual alarm (not shown) at step 38. In this manner, the eye monitor 10 provides for a continuous and real time analysis of the user's eye (or eyes) 17 and in particular the pupil of the (or each) eye, to determine whether the user (not shown) has been exposed to harmful chemicals and without requiring a user (not shown) to break away from their normal duties or to cover their eyes 17.

Referring to FIG. 5, wherein like references have been used to indicate similar integers to those described with reference to FIGS. 1 to 4, there is illustrated a helmet mountable eye monitor 10 according to another embodiment of the present invention.

The eye monitor 10 comprises a tracking arrangement 18, which is arranged initially to locate a centre position of a pupil 19 of one or both of the eyes 17 of a wearer and subsequently to autonomously track the movement of the eyes 17 of the wearer, and in particular movement of the pupils 19 of the wearer's eyes 17. The following description will refer to the tracking and monitoring the pupil 19 of one of the wearer's eyes 17, however, the skilled reader will recognise that the monitor of the present invention could also be used to track and monitor the pupil 19 of each of the wearer's eyes 17. The tracking arrangement 18 is mounted at a suitable location on a helmet, not illustrated, to be donned by a wearer 40. The helmet includes a visor arrangement 42 arranged to be located in front of the eye or eyes 17 of the wearer 40. The visor arrangement 42 is arranged to allow the wearer, in this case a pilot of an aircraft, to view a forward scene through the visor arrangement. The visor arrangement 42 can also be arranged to present an image to overlay the forward scene viewed by the wearer 40. The image overlay can be conformal with the forward scene.

The tracking arrangement 18 comprises a transmitter 20, which is arranged to transmit an infrared probe signal substantially upon the wearer's eye 17 and a receiver 21, such as an infrared camera or sensor, which is arranged to receive an infrared signal which becomes reflected off the wearer's eye 17. The tracking arrangement 18 further comprises signal steering means (not shown) which is arranged to suitably direct the signal upon the wearer's eye 17 and to scan the probe signal across the wearer's eye 17.

The eye monitor 10 further comprises a processor 22 for processing signals received from the receiver 21 and a control unit 23 for controlling the operation of the transmitter 20 and receiver 21. The processor 22 and control unit 23 are communicatively coupled with the tracking arrangement 18 and may be disposed within a cockpit (not shown) or upon the helmet (not shown). In the embodiment illustrated in FIG. 5, the processor 22 and control unit 23 are arranged in communication with the tracking arrangement 18 via a wireless communication link 24. The eye monitor 10 may further comprise a light sensor 25 mounted within the helmet arranged to monitor the ambient light conditions, and is communicatively coupled with the processor 22 via the wireless communications link 24, for example.

The operation of the eye monitor 18 is substantially as that described with reference to FIGS. 3 and 4, above.

Referring to FIG. 6, wherein like references have been used to indicate similar integers to those described with reference to FIGS. 1 to 4, there is illustrated an eye monitor 10 according to another embodiment of the present invention.

The eye monitor 10 comprises a tracking arrangement 18, which is arranged initially to locate a centre position of a pupil 19a, 19b or 19c of one or both of the eyes 17a, 17b or 17c of a plurality of subjects 50a to 50c to be monitored and subsequently to autonomously track the movement of the eyes 17a to 17c of the subjects 50a to 50c, and in particular movement of the pupils 19a to 19c of the eyes 17a to 17c of the monitored subjects 50a to 50c. The following description will refer to the tracking and monitoring the pupil 19 of one of the subjects 50a and associated eye 17a, however, the skilled reader will recognise that the monitor of the present invention could also be used to track and monitor the pupil 19a to 19c of each of the subjects 50a to 50c in turn. The tracking arrangement 18 is mounted at a suitable location to provide a field of view of the subjects 50a to 50c, for example the interior of a vehicle or public space, not illustrated.

The tracking arrangement 18 comprises a transmitter 20, which is arranged to transmit an infrared probe signal substantially upon the subject's 50a eye 17a and a receiver 21, such as an infrared camera or sensor, which is arranged to receive an infrared signal which becomes reflected off the subject's 50a eye 17. The tracking arrangement 18 further comprises signal steering means (not shown) which is arranged to suitably direct the signal upon the subject's eye 17a and to scan the probe signal across the subject's eye 17a.

The eye monitor 10 further comprises a processor 22 for processing signals received from the receiver 21 and a control unit 23 for controlling the operation of the transmitter 20 and receiver 21. The processor 22 and control unit 23 are communicatively coupled with the tracking arrangement 18 and may also be disposed within the interior of the vehicle (not shown) or public space (not shown). In the embodiment illustrated in FIG. 6, the processor 22 and control unit 23 are arranged in communication with the tracking arrangement 18 via communication links 52. The eye monitor 10 may further comprise a light sensor 25 mounted within the interior of the vehicle or public space which is arranged to monitor the ambient light conditions, and is communicatively coupled with the processor 22 via a communications link 54.

The operation of the eye monitor 18 is substantially as that described with reference to FIGS. 3 and 4, above.

It will be understood by a person skilled in the art that the embodiments described herein are intended as examples only and that the separate integers of each embodiment may be combined with features of the other embodiments to provide an eye monitor, head or helmet mountable eye monitor or vehicle mountable eye monitor according to the invention as claimed.

1. An eye monitor for monitoring the size of a pupil of an eye of a monitored subject, the monitor comprising:
an infra-red transmitter for transmitting an infrared radiation probe signal substantially upon an eye of a subject;
an infrared sensor for sensing variations in intensity of the infrared radiation reflected from an eye of a subject and outputting a sensor signal in accordance with sensed variations;
a processor arranged to receive said sensor signal and determine a location of a pupil of a subject dependent on the sensed variations; and
signal steering means arranged to scan a probe signal across an eye of a subject responsive to said processor to direct the probe signal at a pupil of the subject, wherein the processor is arranged to determine a size of the pupil dependent on the sensed variations.

2. (canceled)

3. An eye monitor according to claim 1, wherein the processor is configured to determine the size of a pupil at intervals over a monitoring period.

4. An eye monitor according to claim 3, wherein a frequency of the intervals is dependent on a velocity of the subject.

5. An eye monitor according to claim 1, wherein the processor is configured to determine the size of a pupil substantially continuously over a monitoring period.

6. An eye monitor according to claim 4, wherein the monitoring period is selected for monitoring a subject over a risk period during which the subject is potentially exposed to harmful ambient agents.

7. An eye monitor according to claim 1, comprising:
a memory for storing calibrated pupil size, and wherein the processor is arranged to compare a determined pupil size with said calibrated size for determining exposure to harmful ambient agents when the determined size is inconsistent with the calibrated pupil size.

8. An eye monitor according to claim 1, wherein an intensity of infrared radiation reflected from a pupil of a subject is relatively low and an intensity of infrared radiation reflected from a region of an eye around the pupil of the subject is relatively high, and the processor is configured to determine a location of the pupil by determination of a location of said relatively low reflected variation and to determine a size of the pupil by determination of a distance in at least one dimension across the eye of said relatively low reflected radiation.

9-10. (canceled)

11. A head or helmet mountable eye monitor including an eye monitor according to claim 1, wherein the eye monitor is mounted upon a frame to support the monitor in position relative to a monitored eye.

12. A head or helmet mountable eye monitor according to claim 11, wherein the frame is arranged to support an optical element in front of one or both eyes of a subject.

13. A vehicle mountable eye monitor in combination with the eye monitor of claim 1, wherein the eye monitor is mounted to a structure of a vehicle.

14. A method for monitoring the size of a pupil of an eye of a monitored subject, the method comprising:
transmitting an infrared radiation probe signal substantially upon the eye of the subject;
sensing variations in intensity of the infrared radiation reflected from the eye of the subject and outputting a sensor signal in accordance with the sensed variations;
determining a location of a pupil of a subject dependent on the sensed variations;
scanning the probe signal across the eye of the subject responsive to the determined location of the pupil of the subject; and
determining the size of the pupil dependent on the sensed variations.

15. A method according to claim 14, wherein the intensity of the infrared radiation reflected from the pupil of the subject is relatively low and the intensity of the infrared radiation reflected from a region of the eye around the pupil of the subject is relatively high, and the method comprises:
determining a location of the pupil by determining a location of said relatively low reflected variation and determining the size of the pupil by determining a distance in at least one dimension across the eye of said relatively low reflected radiation.

16. A method according to claim 14, comprising:
detecting exposure of a subject to harmful ambient agents, by determining the size of a pupil of a subject.

17. A method according to claim 16, comprising:
comparing the determined pupil size with a calibrated acceptable pupil range.

18-19. (canceled)
